(19) United States
(12) Patent Application Publication
(10) Pub. No.: US 2024/0215975 A1
(43) Pub. Date: Jul. 4, 2024

(54) CONTINUOUS MENISCUS SUTURE METHOD

(71) Applicants: José Leonardo Rocha DE FARIA, Rio de Janeiro (BR); Cristiano Siqueira Pereira, Juiz de Fora - MG (BR); José Alencar Marques Junior, Juiz de Fora - MG (BR)

(72) Inventors: José Leonardo Rocha DE FARIA, Rio de Janeiro (BR); Cristiano Siqueira Pereira, Juiz de Fora - MG (BR); José Alencar Marques Junior, Juiz de Fora - MG (BR)

(21) Appl. No.: 18/148,128

(22) Filed: Dec. 29, 2022

(51) Int. Cl. A61B 17/04

(52) U.S. Cl. CPC A61B 17/0485; A61B 17/0491; A61B 2017/06052

(57) ABSTRACT

The present invention refers to a continuous meniscus suture, the chain method, performed with a suture device comprising a cannula having a pointed end and a slidable inner rod with a hook at the end, with the steps of: a) accessing the joint; b) perforating the meniscus with the suture device in a first region of the injury; c) capturing a suture thread with the hook of the suture device; d) pulling the suture thread forming a loop inside the joint and positioning the loop in a second region of the injury on the side opposite the prior perforation site of the meniscus; e) releasing the thread of the hook of the device; f) perforating the meniscus with the end of the device in the second region of the injury, passing the cannula of the suture device inside the loop positioned in step d); g) capturing again the suture thread outside the joint; h) recoiling the device inside the joint, bringing the new loop into the joint; i) positioning the new loop in the desired position, crossing the injury about 4 mm prior to the last one, and introducing the device crossing the injury; j) carrying out steps f) to i) in sequence until the entire extension of the injury is covered; k) after covering the entire extension, with the device inside the joint, perforate the meniscus taking the last loop formed by the suture thread fastened to the hook out of the joint; l) releasing the loop in the outer region of the joint; m) closing the suture passing one end of the thread inside the loop and pulling the ends of the suture thread, so as to tighten the loops disposed in the inner region of the joint.

The present invention refers to a meniscus suture method that can be carried out continuously, using loops passed over loops.

### DESCRIPTION OF THE STATE OF THE ART

Lesions of the meniscus are a type of injury in the knee joint which, in most cases, requires surgical intervention by arthroscopy for treatment. The procedure consists of suturing the torn meniscus so that healing can occur. Various suturing techniques used in repairing a meniscal injury are known in the state of the art.

The most widespread technique, deemed to be the “gold standard” in the field of meniscus surgeries, is the ‘inside-out’ suture, performed with a device having a rigid cannula with angulation of about 15° at the end through which a needle passes having a lumen at its end opposite the perforating one that transports a suture thread. The cannula is introduced into the joint and positioned near the injury, using its curvature to direct the needle correctly. The needle is introduced, passing inside the injured region of the meniscus so that the suture thread passes inside the injury. Thus, one end of the suture thread is transported through the inside of the meniscus and remains accessible to the surgeon, while at the other end it remains inside the cannula. This sequence of steps is repeated at another point so that the other end of the thread passes through the meniscus and then a stitch can be sown. For each additional stitch to be sown, the full sequence of steps above must be reproduced, until the entire injury is covered and duly sutured.

Although this technique is considered the “gold standard”, it clearly bears shortcomings that make it hard to be carried out. The main problem noted is the time of execution of the suture. Each stitch needs to be individually executed and closed, without continuity of movement, leading to a slow and tedious process.

Another known technique is the so-called ‘outside-in’. This technique is performed with two needles and the assistance of pincers to capture the thread, but it does not enable stitches to be sown in sequence in a continuous manner either. The suture threads are passed with the needles and caught by the pincers to form the stitches, and then closed with knots. Additionally, it is a decidedly less precise and relatively complex technique, as there is little accuracy in the place where the needles are introduced and the place where the needles should penetrate the meniscus, multiple perforations being necessary until the suitable positioning of the needles is achieved, in addition to using conventional needles or needles used in anesthetic procedures to carry out said technique, a fact that causes the surgeon hardship in handling them.

Lastly, the ‘all-inside’ is a technique performed with a specific device and with a single surgical access. The device has two plates and a suture thread with pre-molded knots. A first triggering of the device in a first region of the injury releases the first plate and a second triggering in another region on the opposite side of the injury releases the second plate. Upon withdrawing the device with the plates disposed, the suture thread is pulled and the pre-molded knots join the two plates forming a stitch. For each stitch, a new device is needed, so this technique does not permit continuity of movement, in addition to being rather expensive due to the quantity of devices used in a single injury.

### OBJECTIVES OF THE INVENTION

The objective of the present invention is to provide a meniscus suture method which may be performed continuously, assuring greater agility in the surgery time to assist meniscal healing.

### BRIEF DESCRIPTION OF THE INVENTION

The objectives of the invention are achieved by means of a continuous suture method on the meniscus carried out with a suture device comprising a cannula having a pointed end and a slidable inner rod with a hook at the end. The method is carried out inside the knee joint and comprises the steps of:
a) performing the posteromedial or posterolateral access in the knee;
b) perforating the meniscus with the pointed end of the suture device in a first region of the injury;
c) capturing a suture thread with the hook of the suture device;
d) pulling the suture thread through the perforation forming a loop inside the joint and positioning the loop in a second region of the injury on the opposite side to the previous perforation site of the meniscus;
e) releasing the suture thread of the hook of the suture device;
f) perforating the meniscus with the end of the suture device in the second region of the injury, passing with the cannula of the suture device inside the loop positioned in step d);
g) capturing again the suture thread outside the joint with the hook of the device;
h) recoiling the device inside the joint bringing a new loop into the joint;
i) positioning the new loop in the desired position, crossing the injury about 4 mm prior to the last place introduced and at this point introduce the device near to the injury, crossing the injury;
j) carrying out steps f) to i) in sequence until the entire extension of the injury is covered;
k) after covering the entire extension of the injury, with the device situated inside the joint, perforate the meniscus taking the last loop formed by the suture thread fastened to the hook out of the joint in the surgical approach;
l) releasing the loop in the outer region of the joint;
m) closing the suture passing one end of the thread inside the loop and pulling the ends of the suture thread, so as to tighten the loops disposed in the inner region of the joint.

In a preferred embodiment of the invention, the first iteration of step b) is carried out in the most proximal and posterior region of the injury.

In a preferred embodiment of the method, each hole subsequent to the first hole is spaced 4 mm from the preceding hole.

### BRIEF DESCRIPTION OF THE DRAWINGS

For improved understanding, the characteristics and advantages of the present invention will be presented and described jointly with their respective drawings, which illustrate the following:

FIG. 1 illustrates an embodiment of the device used in performing the method.

FIG. 2a illustrates an exposed hook position of the device.

FIG. 2b illustrates a hook in partially retracted position of the device.

FIG. 2c illustrates a hook in retracted position of the device.

FIG. 3 illustrates a meniscus injury.

FIG. 4 illustrates a preferred position of the first perforation of the meniscus.

FIG. 5 illustrates a first perforation of the meniscus.

FIG. 6 illustrates a capture step of the suture thread.

FIG. 7 illustrates a capture step of the suture thread.

FIG. 8 illustrates the formation of the first loop with the suture thread.

FIG. 9 illustrates the positioning of the first loop.

FIG. 10 illustrates a preferred position of a second perforation of the meniscus.

FIG. 11 illustrates a preferred position of a second perforation of the meniscus.

FIG. 12 illustrates a capture step of a second loop.

FIG. 13 illustrates a capture step of a second loop.

FIG. 14 illustrates a capture step of a second loop.

FIG. 15 illustrates a capture step of a second loop.

FIG. 16 illustrates a pulling step of a second loop into the joint.

FIG. 17 illustrates a preferred position of a second loop.

FIG. 18 illustrates a perforation step in a third position of the meniscus.

FIG. 19 illustrates a capture step of a third loop.

FIG. 20 illustrates a capture step of a third loop.

FIG. 21 illustrates a pulling step of a third loop into the joint.

FIG. 22 illustrates a preferred position of a third loop.

FIG. 23 illustrates a perforation step in a fourth position of the meniscus.

FIG. 24 illustrates a capture step of a fourth loop.

FIG. 25 illustrates a capture step of a fourth loop.

FIG. 26 illustrates a pulling step of a fourth loop into the joint.

FIG. 27 illustrates a preferred position of a fourth loop.

FIG. 28 illustrates a perforation step in a fifth position of the meniscus.

FIG. 29 illustrates a capture step of a fifth loop.

FIG. 30 illustrates a capture step of a fifth loop.

FIG. 31 illustrates a pulling step of a fifth loop into the joint.

FIG. 32 illustrates a preferred position of a fifth loop.

FIG. 33 illustrates a perforation step in a sixth position of the meniscus.

FIG. 34 illustrates a capture step of a sixth loop.

FIG. 35 illustrates a pulling step of a sixth loop into the joint.

FIG. 36 illustrates a preparation for the perforation for closing the suture.

FIG. 37 illustrates a perforation with a loop fastened to the device in the hole position for closing the suture.

FIG. 38 illustrates the release of the loop on the outer region of the injury.

FIG. 39 illustrates a closing step of the suture.

FIG. 40 illustrates a closing step of the suture.

FIG. 41 illustrates a closing step of the suture.

FIG. 42 illustrates a closing step of the suture.

FIG. 43 illustrates a closing step of the suture.

FIG. 44 illustrates a closing step of the suture.

FIG. 45 illustrates a closing step of the suture.

FIG. 46 illustrates a closing step of the suture.

FIG. 47 illustrates a closing step of the suture.

FIG. 48 illustrates a closing step of the suture.

FIG. 49 illustrates a closing step of the suture.

FIG. 50 illustrates a closing step of the suture.

### DETAILED DESCRIPTION OF THE INVENTION

The continuous chain suture method of the meniscus enables the end-to-end closing of a meniscus injury with a single suture thread by a continuous suture movement of the first point of perforation up until the last, finalizing with a single closing knot.

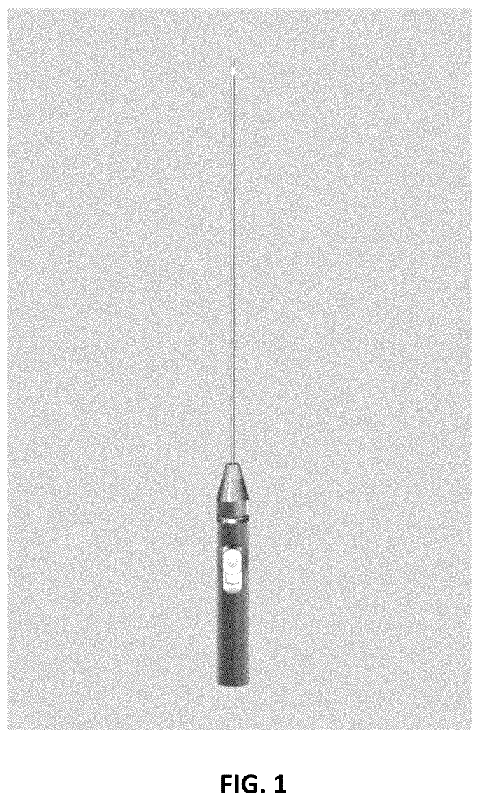

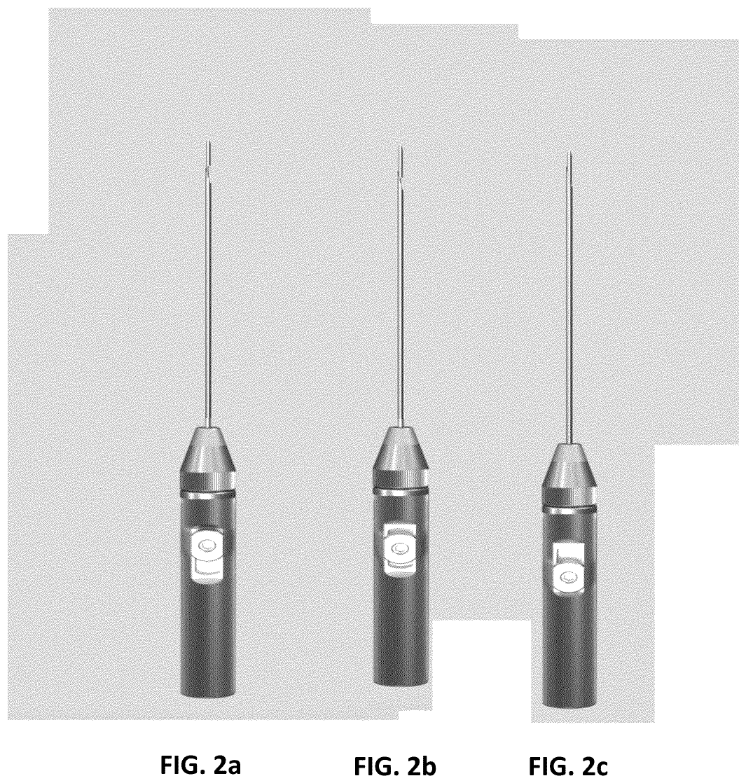

The method is performed with a device with a cable which has a button that slides upwards and downwards, shown in FIG. 1. This button triggers a rod with hook at the end that slides inside a cannula having a pointed end about 1.7 mm thick. When the button is positioned upwards the hook is visible outside the end of the cannula and when the button is positioned downwards it transports the hook inside the cannula, where it can no longer be seen, as shown in FIGS. 2a, 2b and 2c.

After identifying and measuring the size of the injury in the meniscus, a posteromedial or posterolateral access path is made for introducing the meniscus suture device into the joint, in accordance with the location of the injury in the meniscus.

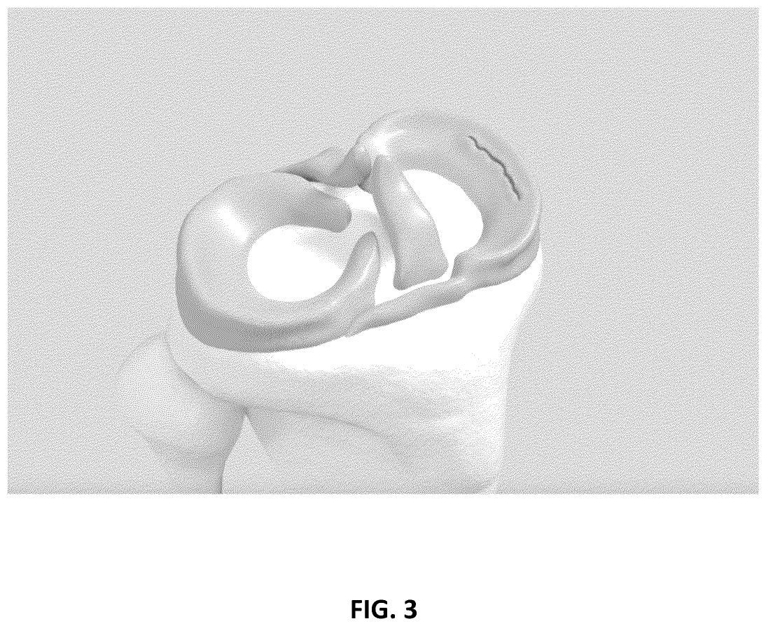

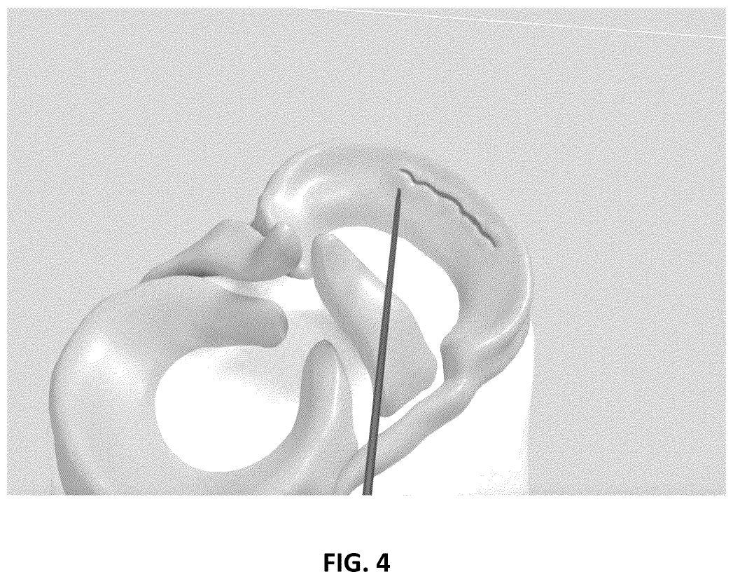

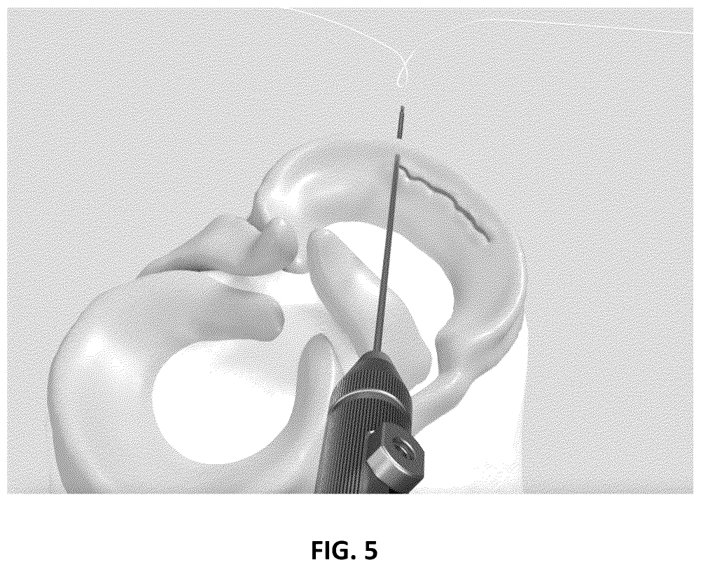

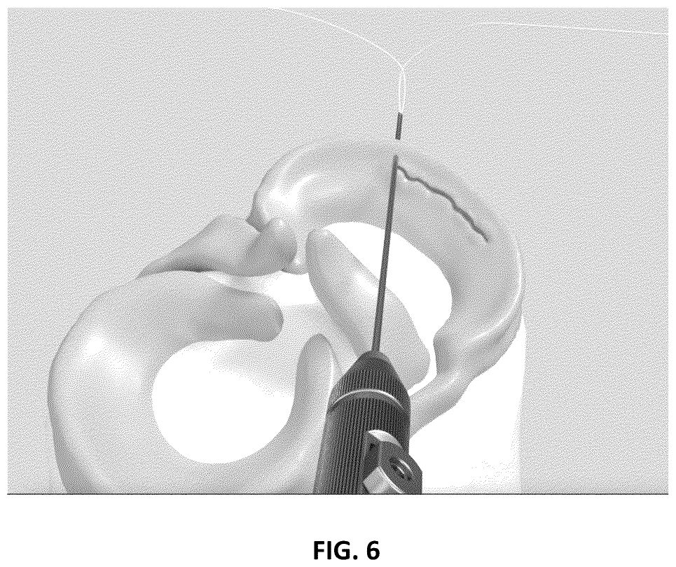

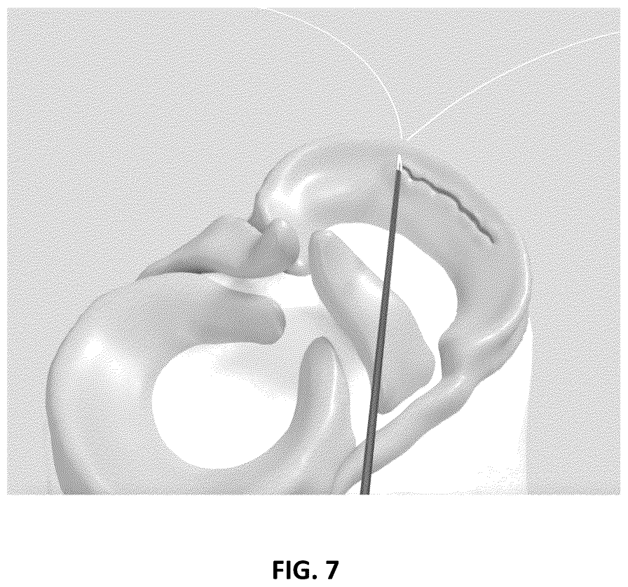

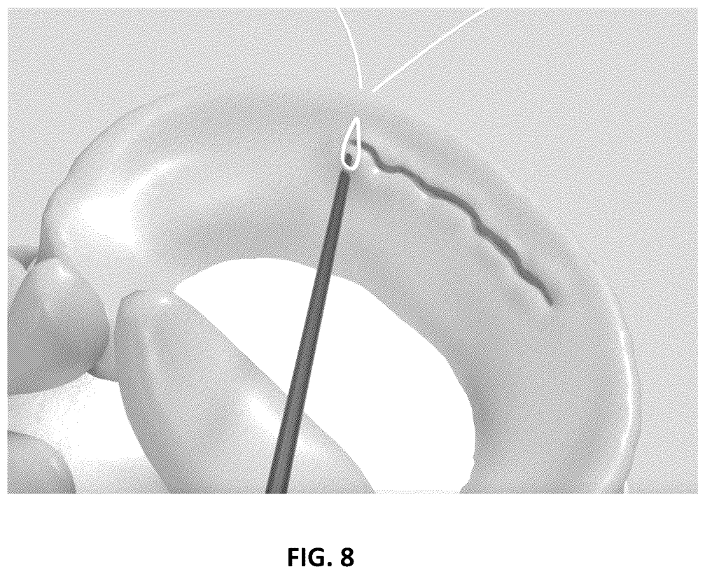

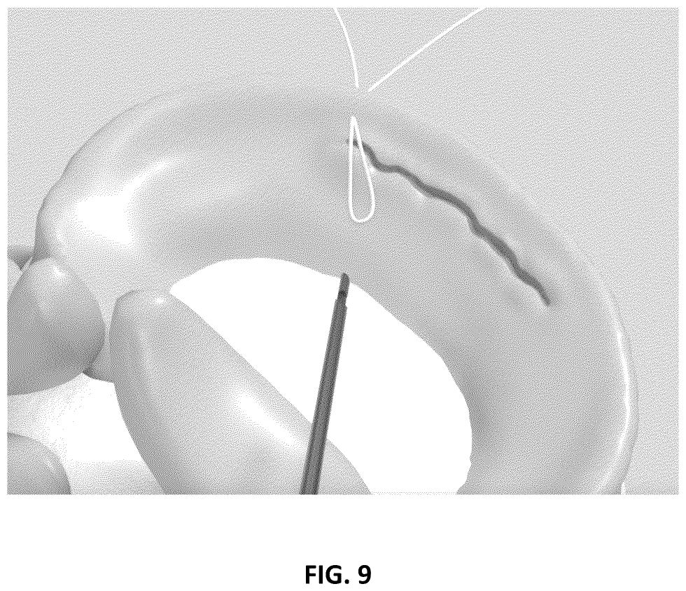

FIGS. 3 to 9 illustrate the making of the first perforation, which marks the start of the suture method. The method begins by perforating the meniscus with the pointed end of the suture device in a first region of the injury, preferably in a more proximal and posterior region of the injury. With the device already traversing the region of the meniscus, a suture thread should be captured with the hook of the suture device and the suture thread pulled through the perforation forming a loop inside the joint. The surgeon should then position the loop in a second region of the injury on the side opposite the preceding hole, preferably at a distance of 4 mm from the preceding hole and release the suture thread of the hook of the suture device.

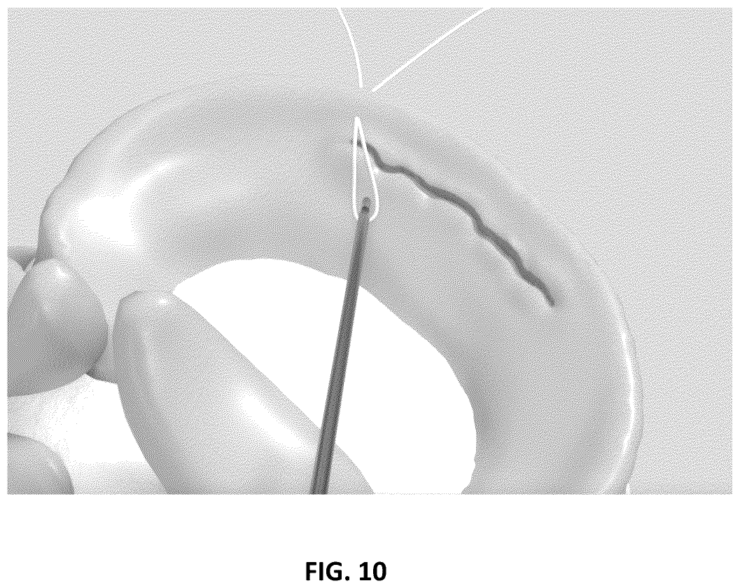

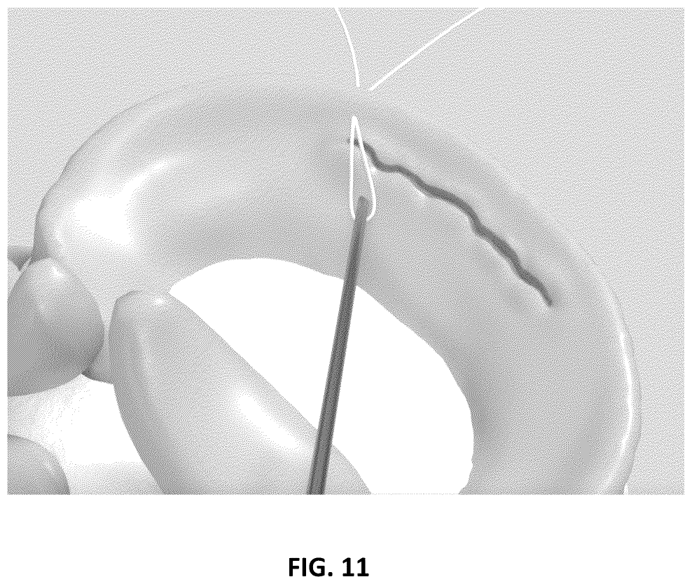

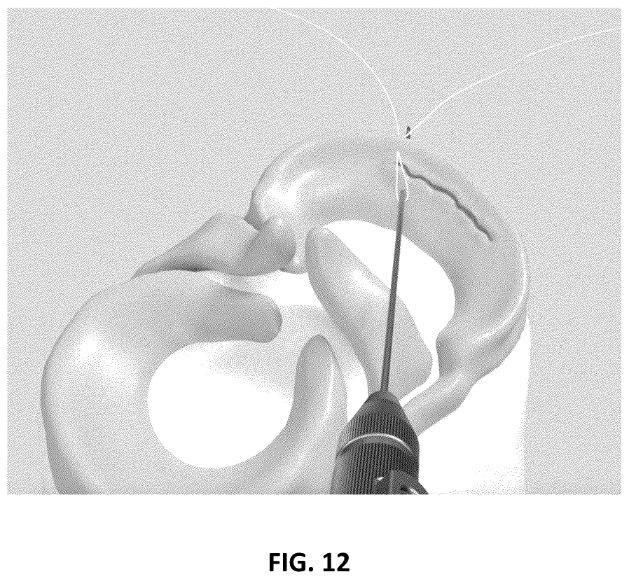

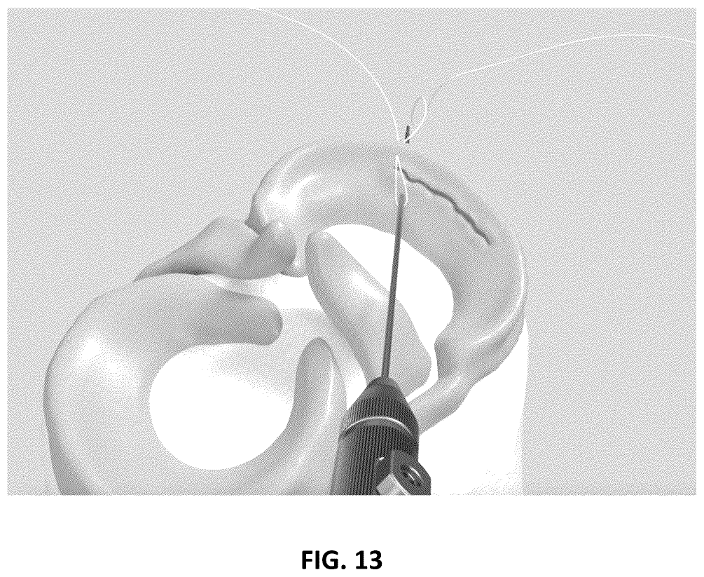

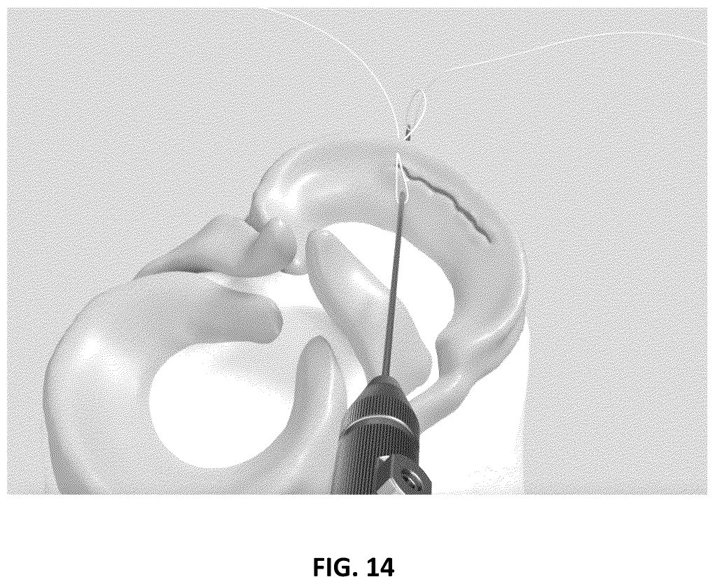

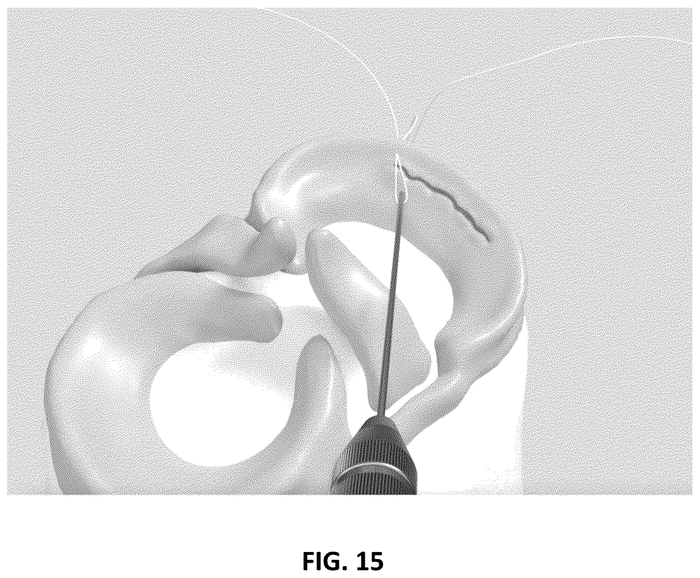

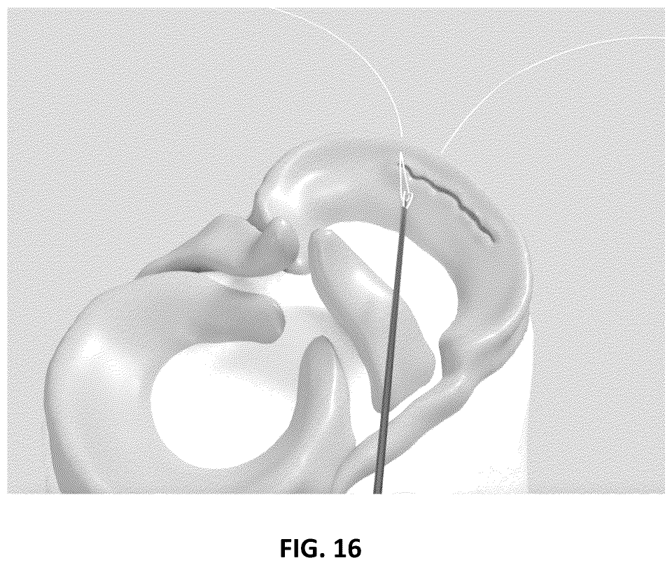

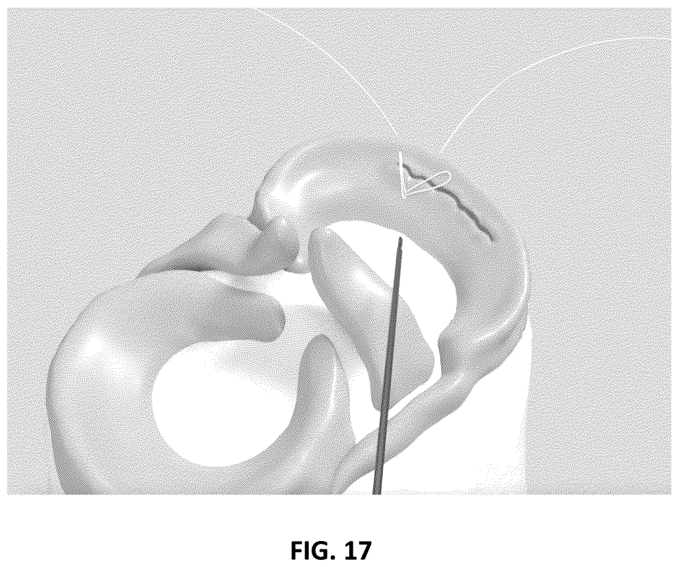

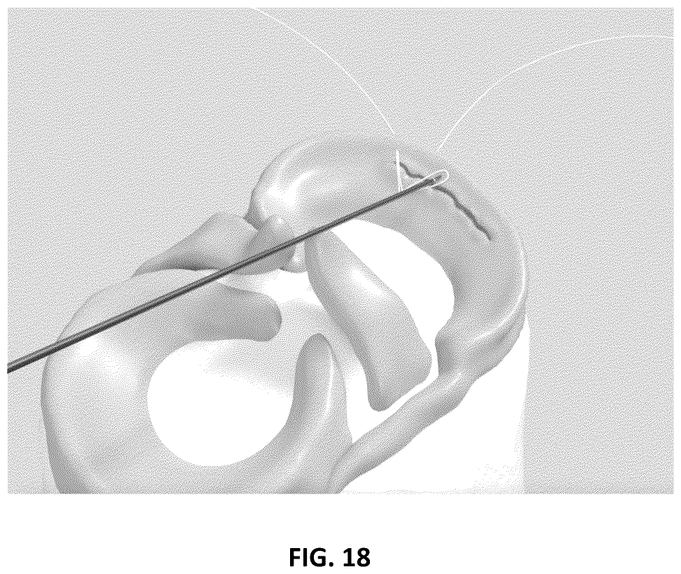

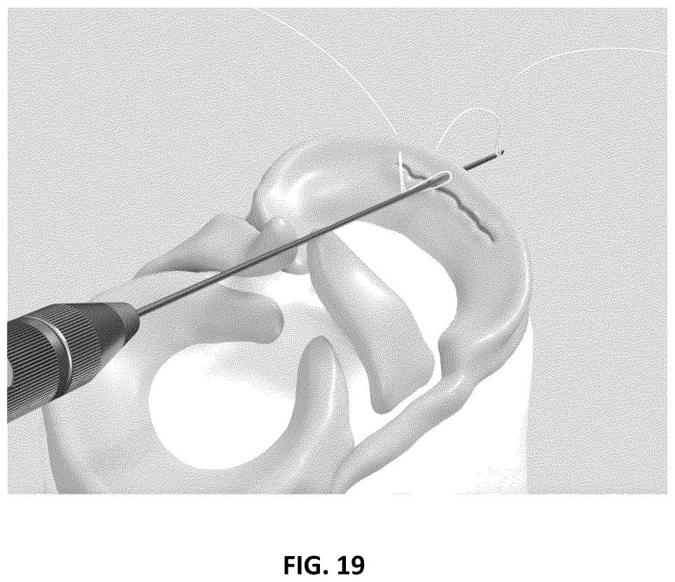

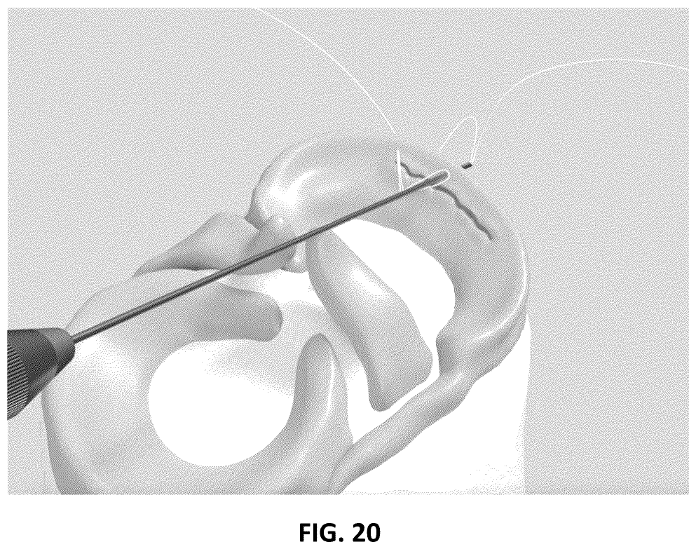

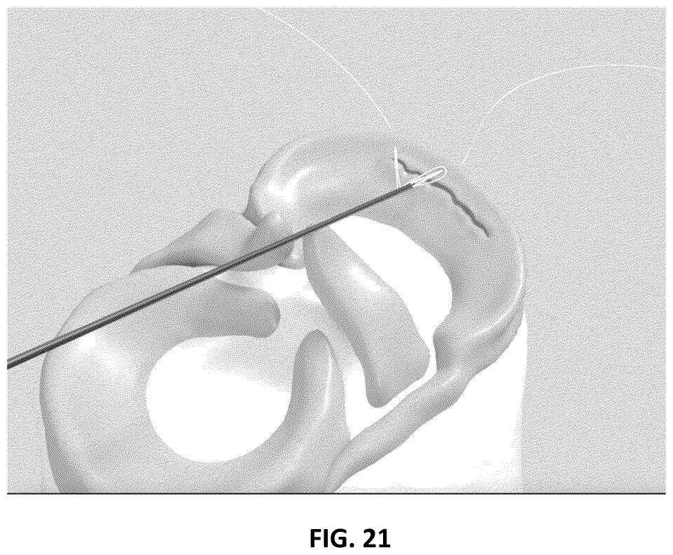

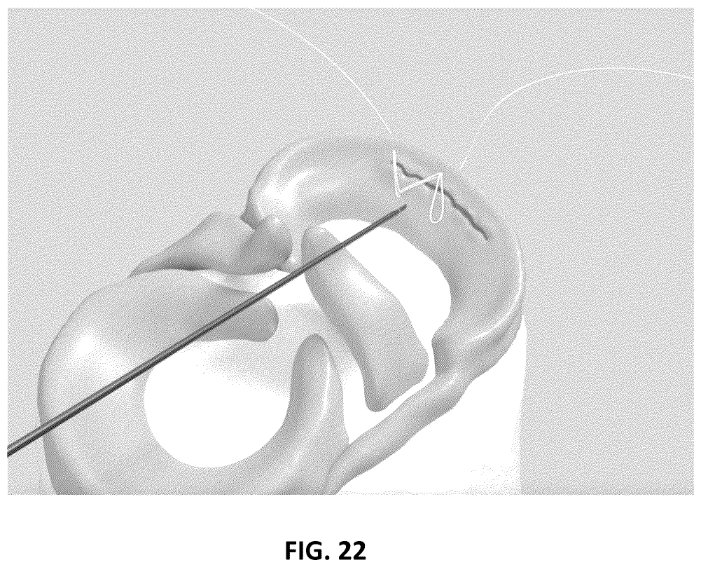

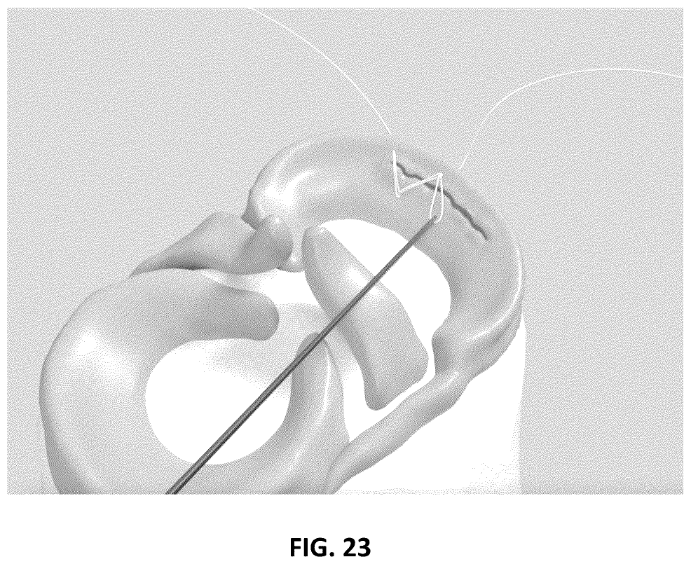

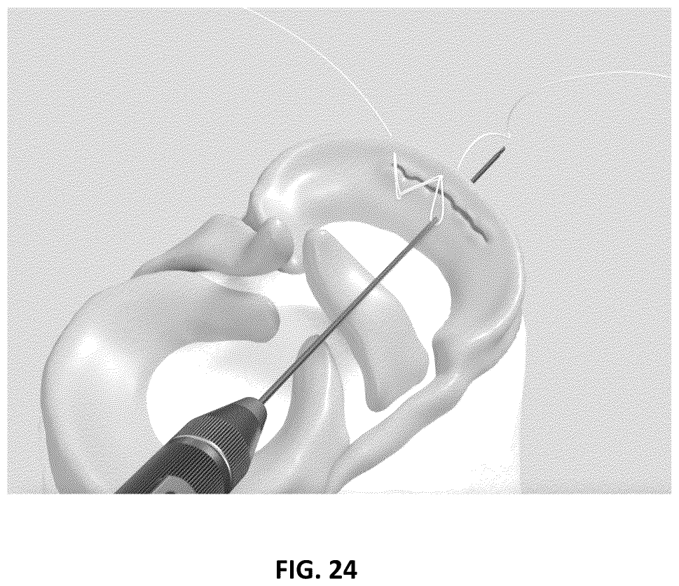

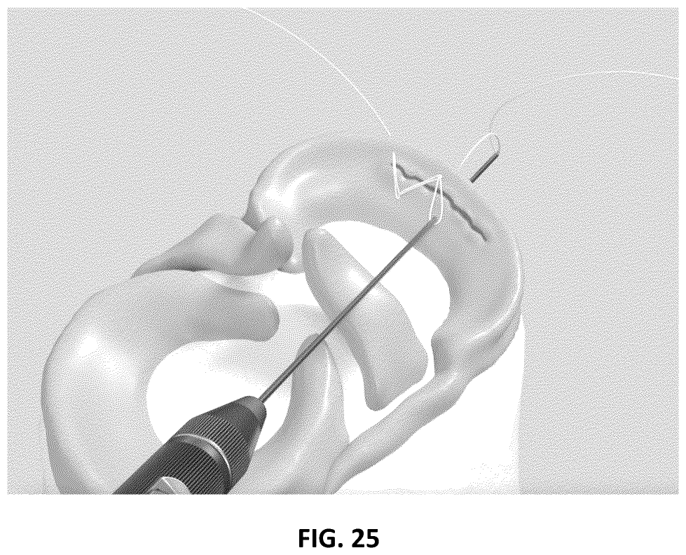

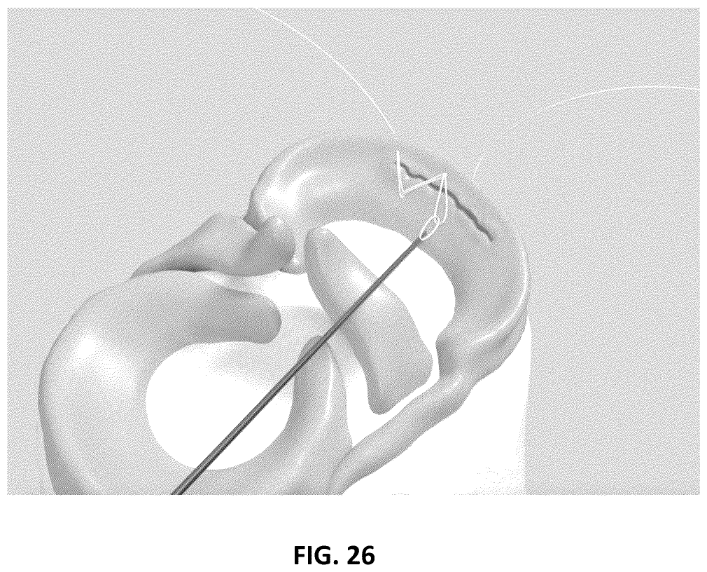

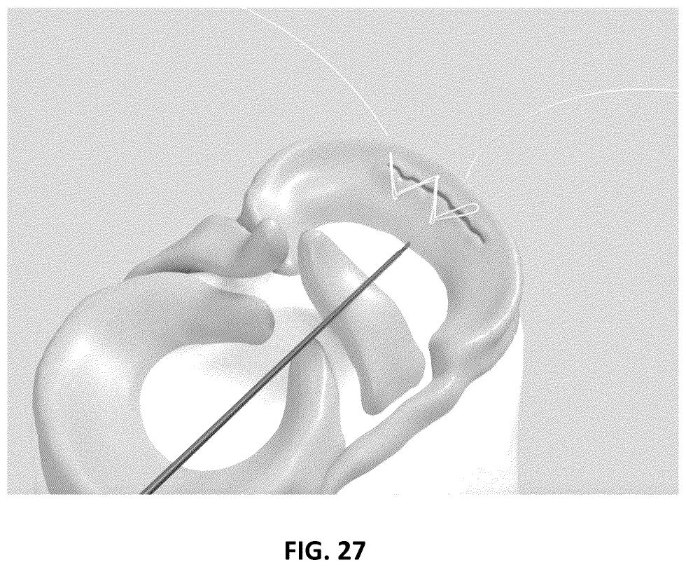

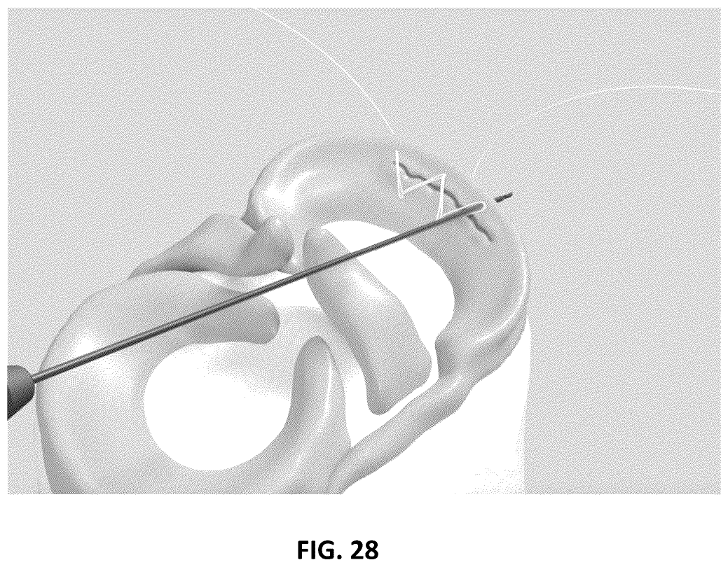

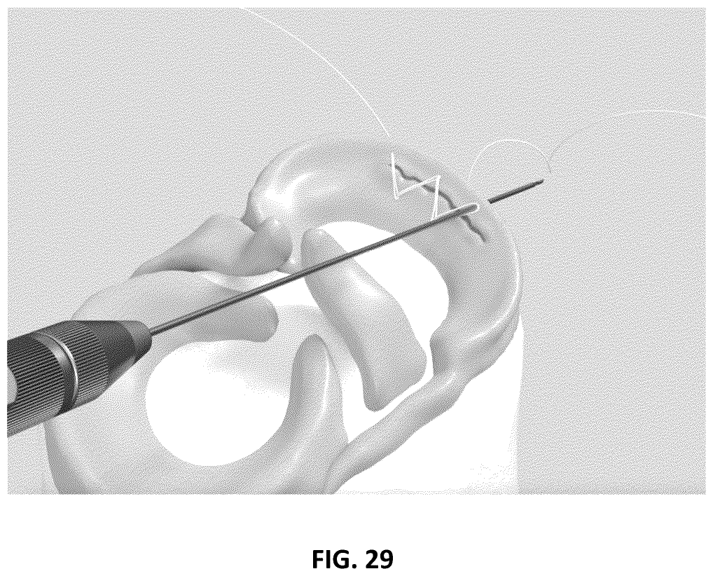

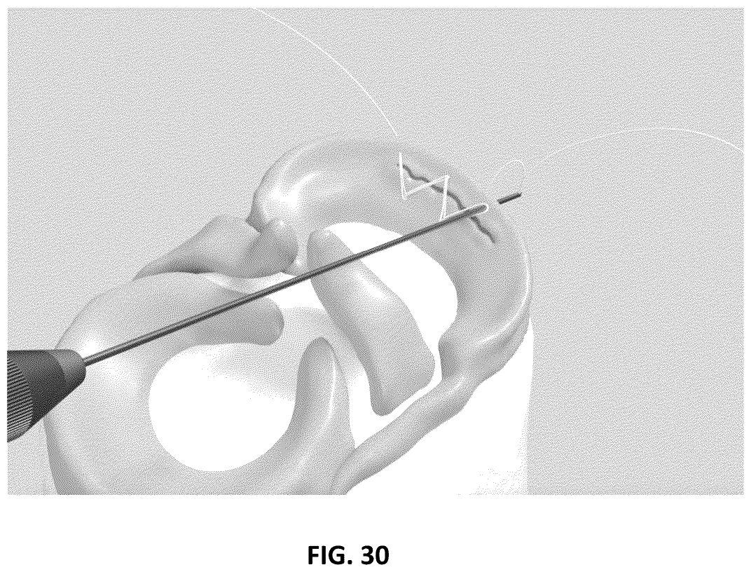

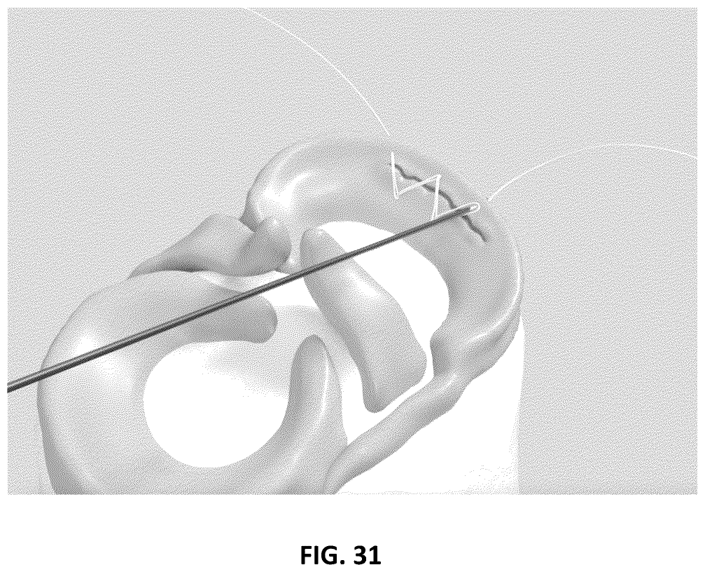

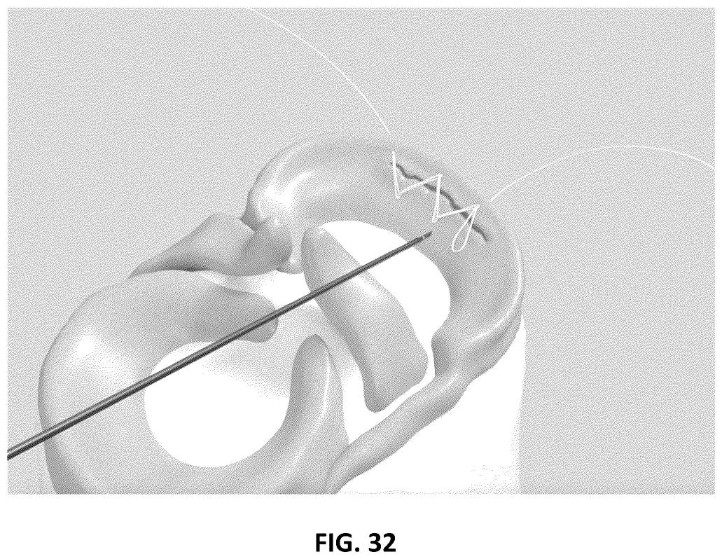

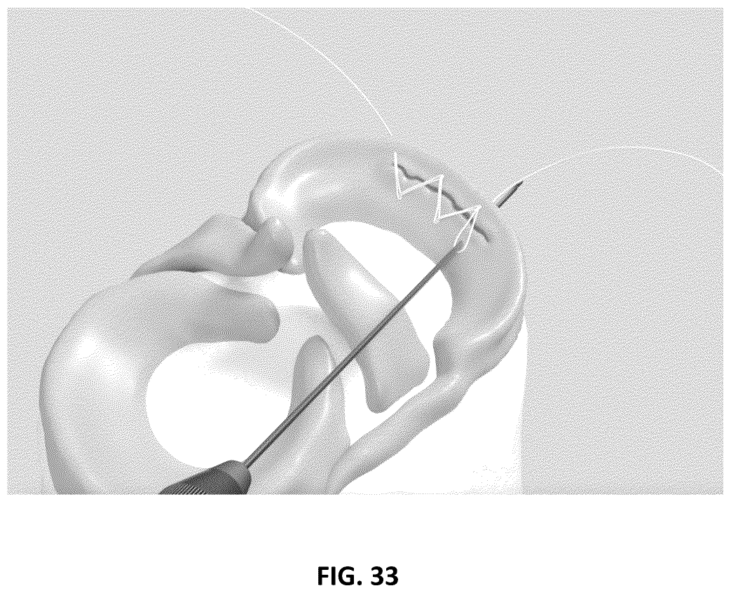

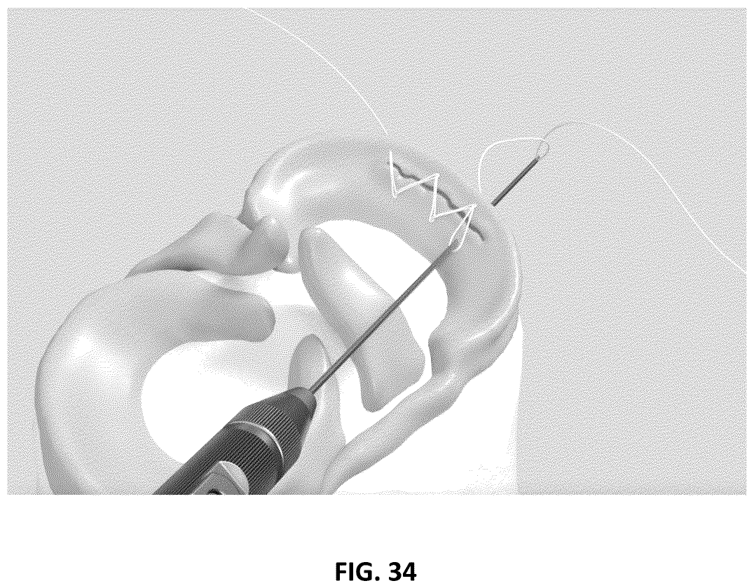

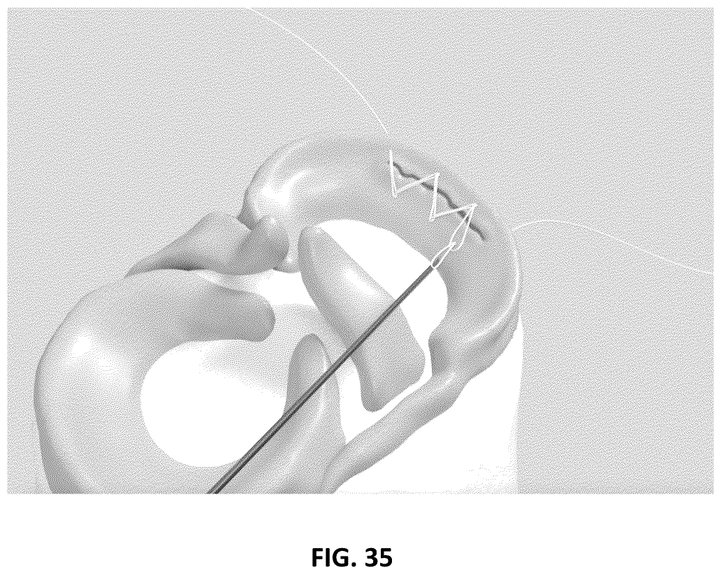

As shown in FIGS. 10 to 17, the surgeon perforates the meniscus in the second region of the injury, passing the cannula inside the loop formed by the suture thread pulled in the preceding hole. The suture thread is again captured with the hook at the end of the suture device by the new access, forming a new loop which is then pulled inside the joint upon recoiling the device inwardly thereof. The loop is positioned on the side opposite the second perforation, that is, on the same side where the first perforation was made, and then released inside the joint. Said sequence of steps is repeated along the entire length of the injury, as can be seen from FIG. 18 to FIG. 35.

Upon reaching the end of the injury, steps are carried out that allow the closure of the continuous suture. The last loop formed should not be released from the hook, as it is used to close the stitch on the outer region of the joint. The surgeon should perforate the meniscus in a region opposite that of the last hole with the loop of the suture thread still fastened on the hook of the suture device and then release the loop on the outer part of the joint. With the ends of the suture thread and the loop on the outer part, the suture is closed, pulling the ends such that the loops formed in the inner region of the joint are tightened and make the closure of the injury in the meniscus.

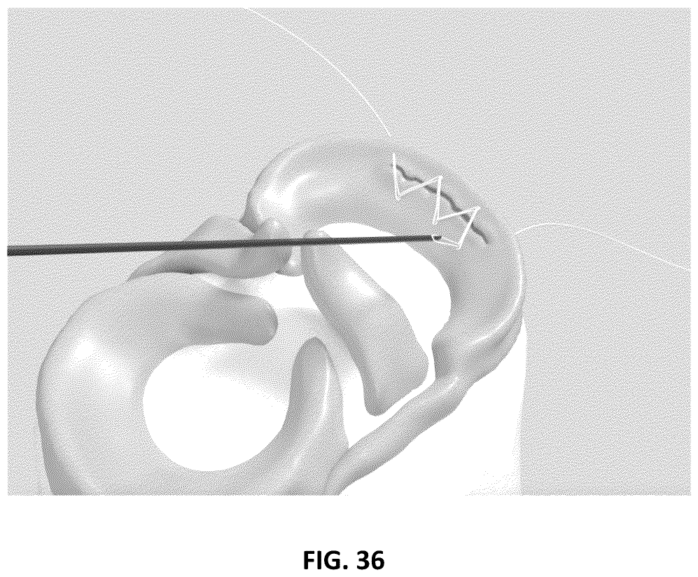

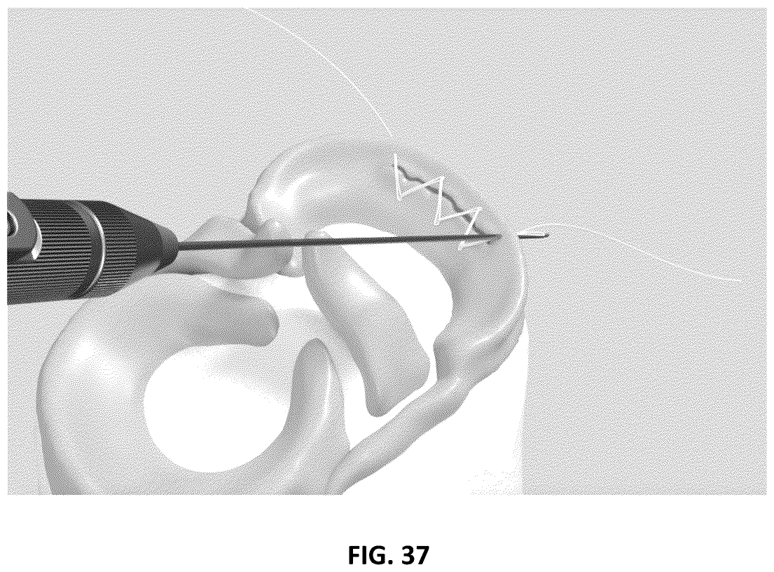

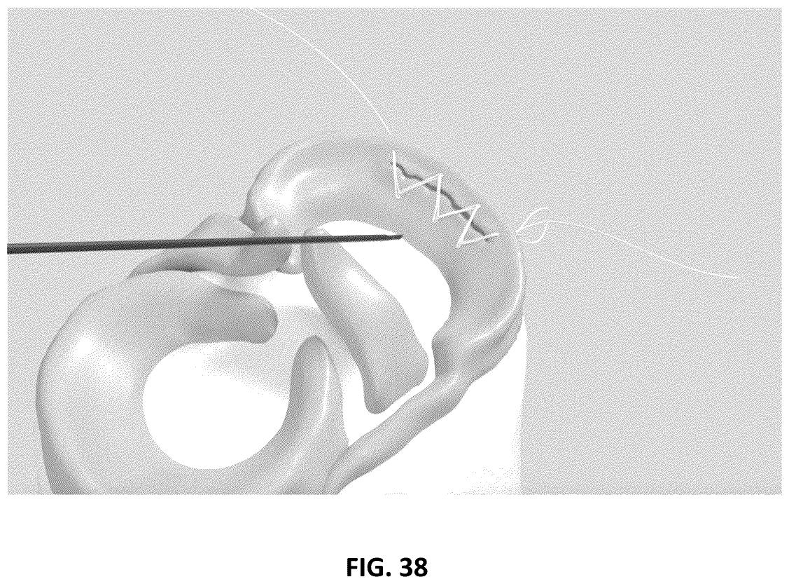

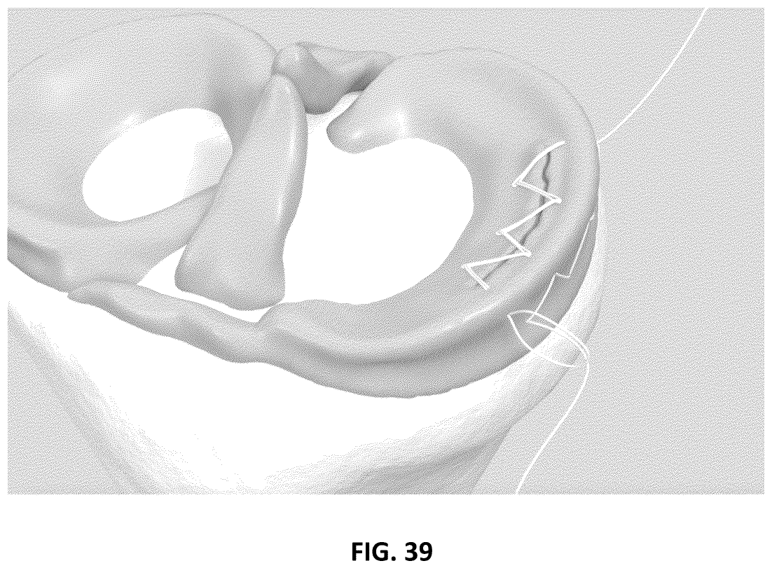

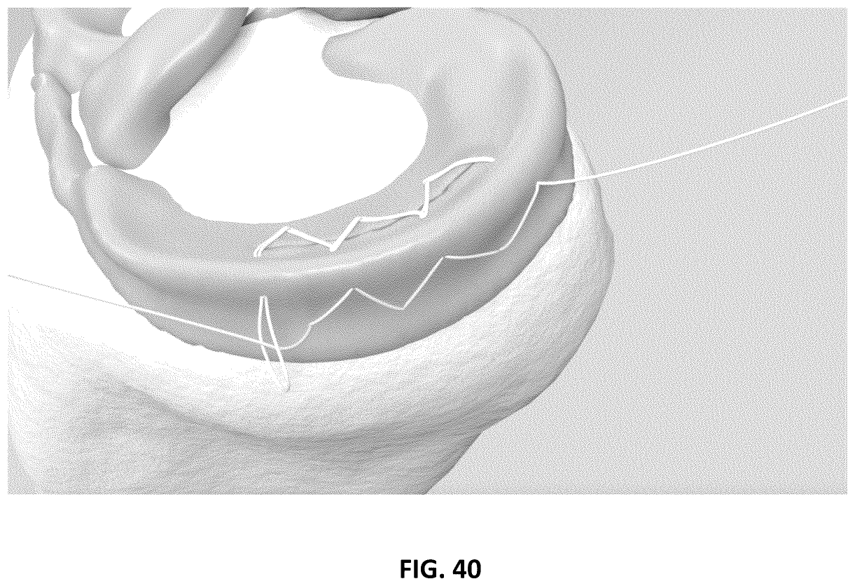

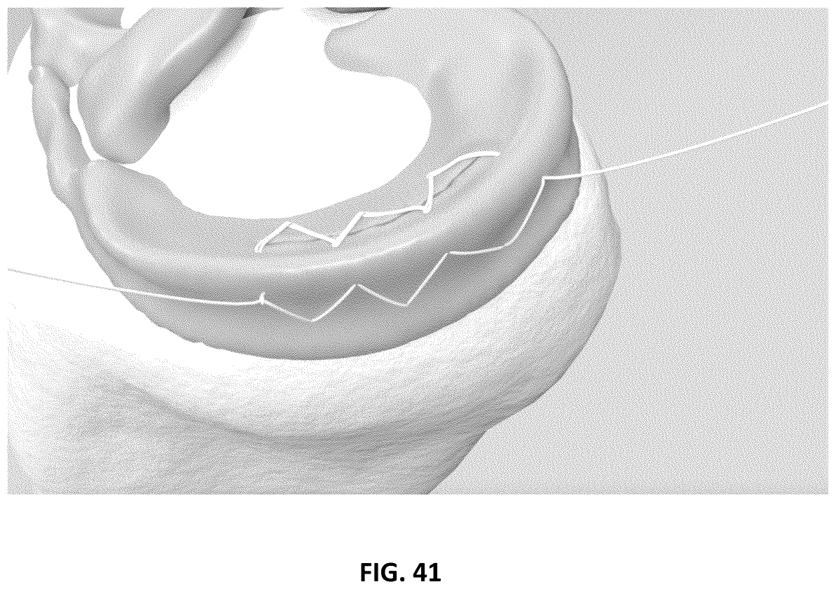

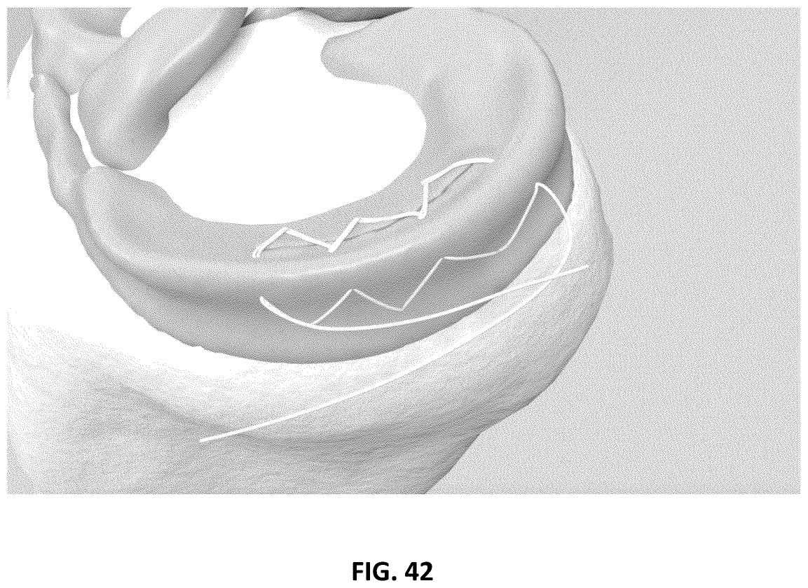

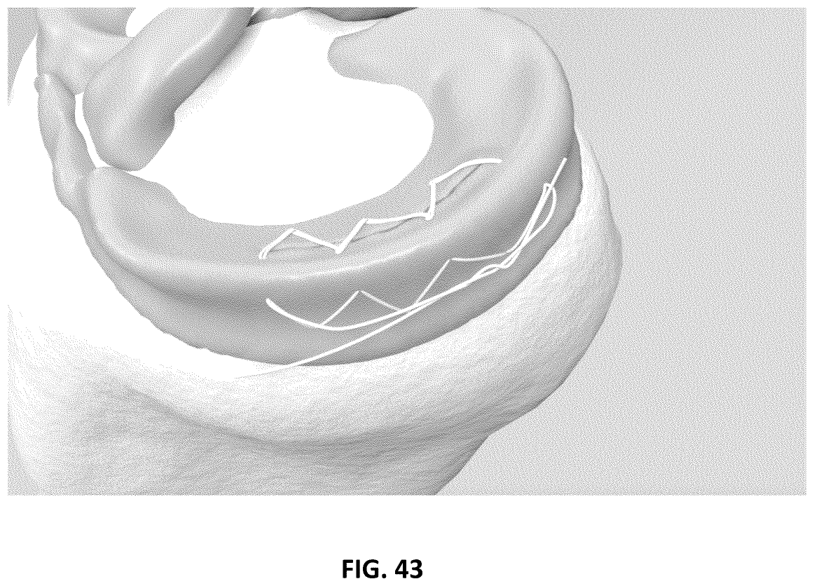

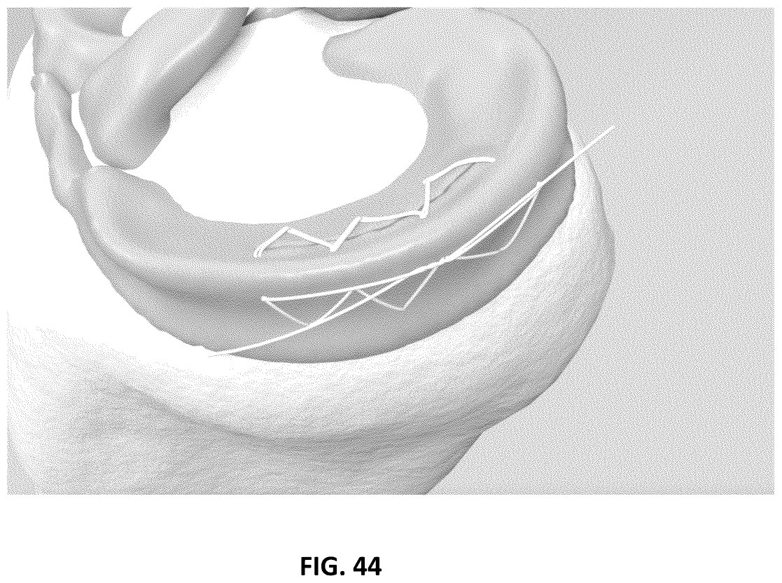

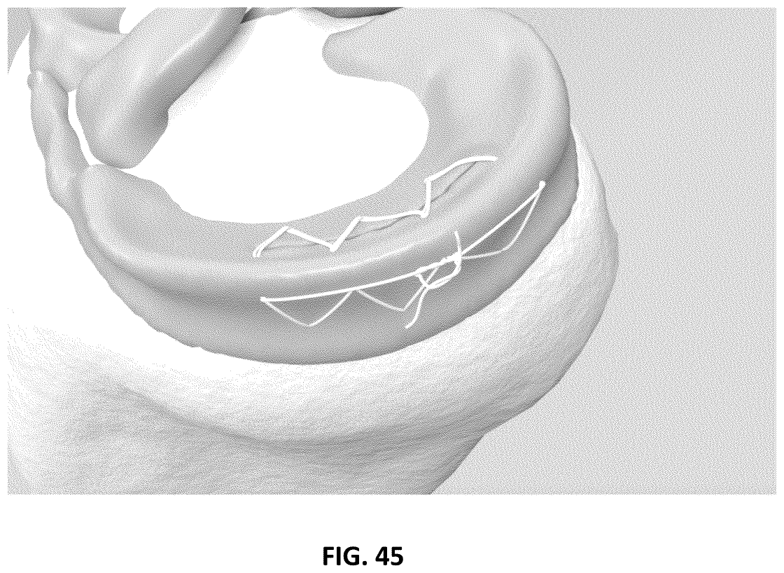

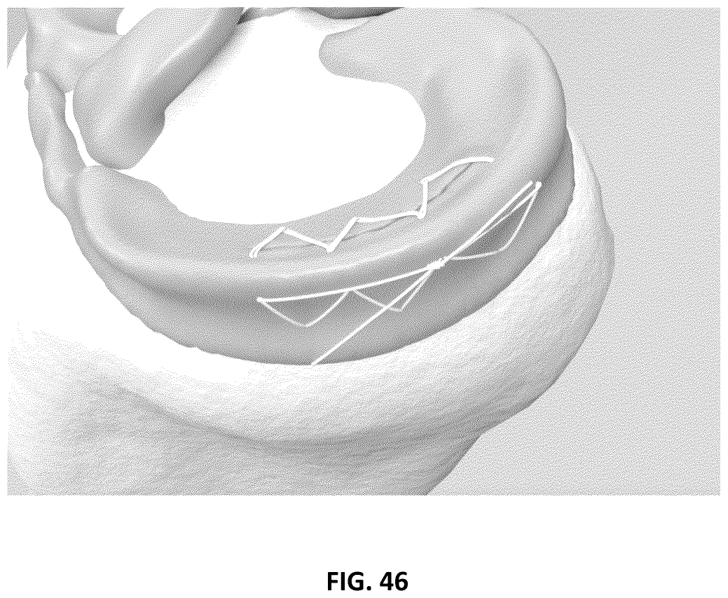

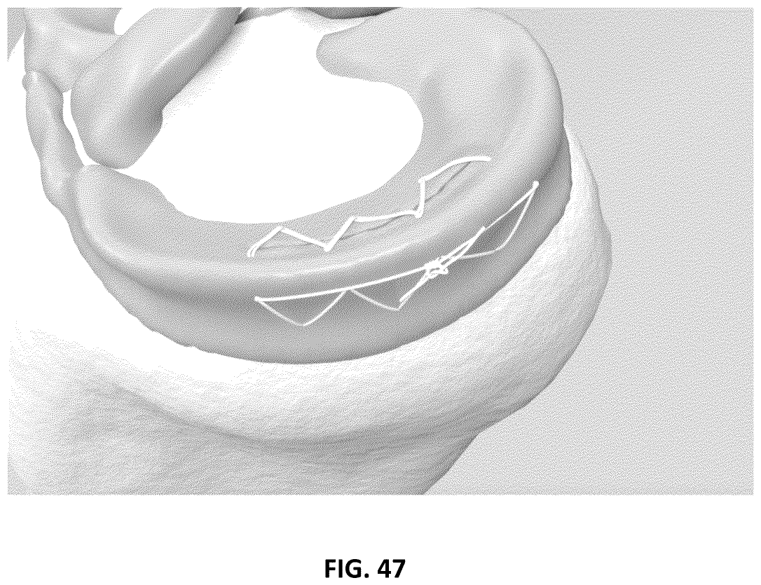

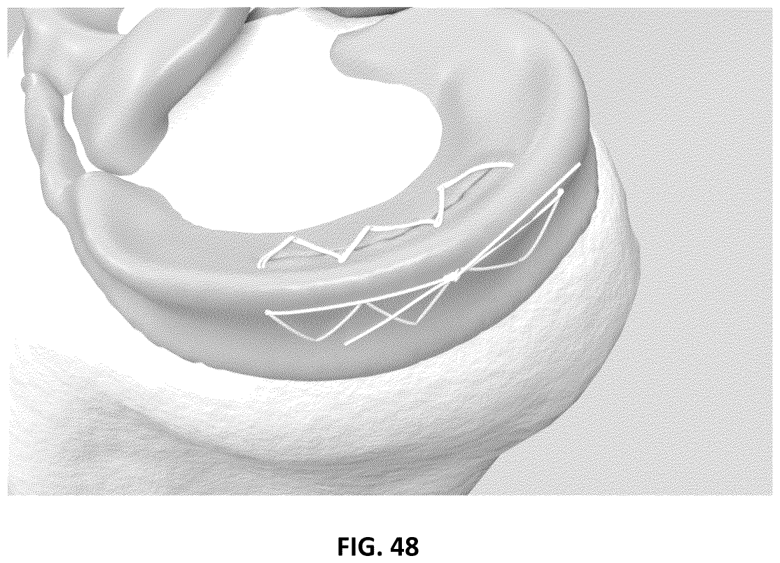

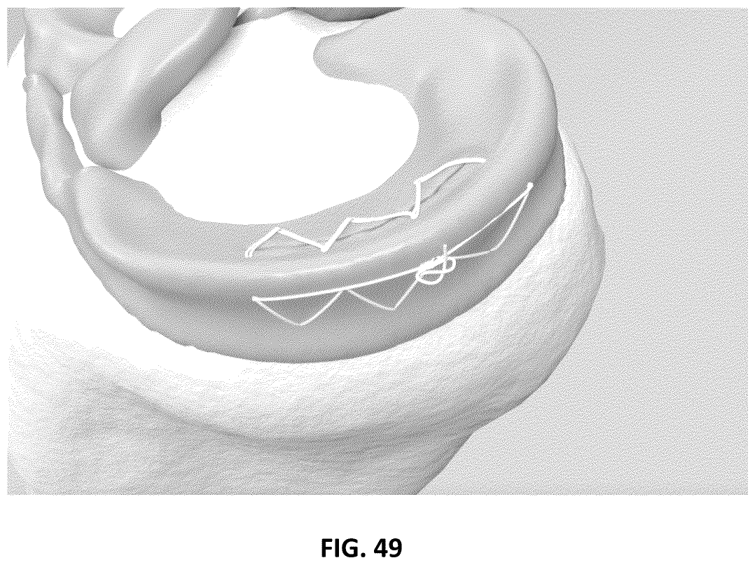

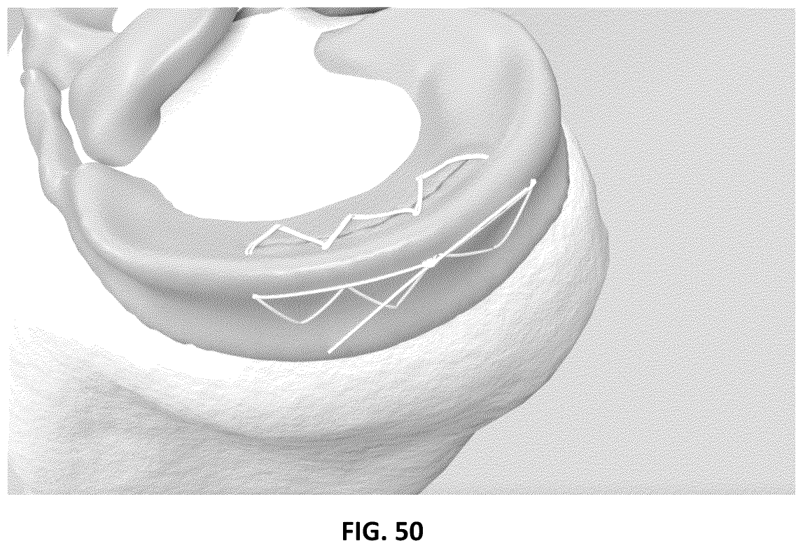

FIGS. 36 to 50 illustrate the closing of the suture, and it is possible to note that all the loops are closed with a single knot in common.

The method according to the present invention is particularly advantageous because it enables the surgeon to make a precise suture of the meniscus in a continuous manner and in less time when compared to the traditional methods, fastening the meniscus with double loops, with fewer knots and with simple equipment.

The differentiating factor of this method is the use of the loop-over-loop configuration, enabling potentially more resistant sutures to be made. The chain suture works such that the strength to bring the tissues together is carried out in double threads. This is how meniscal edges are approximated. The stitches exert traction on the intertwined loops on the chain. This is why there is a lower probability of injuring the meniscus when the knots are tightened, increasing the possibility that the meniscal tissue will heal.

1. A continuous chain meniscus suture method performed with a suture device comprising a cannula having a pointed end and a slidable inner rod with a hook at the end, characterized in that the method is carried out inside the knee joint and comprises the steps of:
a) making a posteromedial or posterolateral access on the joint;
b) perforating the meniscus with the pointed end of the suture device in a first region of the injury;
c) capturing a suture thread with the hook of the suture device;
d) pulling the suture thread through the perforation forming a loop inside the joint and positioning the loop in a second region of the injury on the side opposite the prior perforation site of the meniscus;
e) releasing the suture thread of the hook of the suture device;
f) perforating the meniscus with the end of the suture device in the second region of the injury, passing the cannula of the suture device inside the loop positioned in step d);
g) capturing again the suture thread outside the joint with the hook of the device;
h) recoiling the device inside the joint, bringing a new loop into the joint;
i) position the new loop in the desired position, crossing the injury about 4 mm prior to the last site introduced and introduce the device near to the injury, crossing the injury;
j) carrying out steps f) to i) in sequence until the entire extension of the injury is covered;
k) after covering the entire extension of the injury, with the device inside the joint, perforate the meniscus taking the last loop formed by the suture thread fastened to the hook outside the joint in the surgical approach;
l) releasing the loop in the outer region of the joint;
m) closing the suture passing one end of the thread inside the loop and pulling the ends of the suture thread, so as to tighten the loops disposed in the inner region of the joint.

2. The method according to claim 1, characterized in that the first iteration of step b) is performed in the most proximal and posterior region of the injury.

3. The method according to claim 1, characterized in that each hole subsequent to the first hole is spaced 4 mm from the preceding hole.
